(19) United States
(12) Patent Application Publication
(10) Pub. No.: US 2019/0021653 A1
(43) Pub. Date: Jan. 24, 2019

(54) MONITORING THE POSTURE OF A USER

(71) Applicant: INTERNATIONAL BUSINESS MACHINES CORPORATION, Armonk, NY (US)

(72) Inventors: ERNESTO ARANDIA, MULHUDDART (IE); ROHINI GOSAIN, MULHUDDART (IE); FEARGHAL O'DONNCHA, GALWAY (IE); EMANUELE RAGNOLI, DUBLIN (IE); SESHU TIRUPATHI, DUBLIN (IE)

(21) Appl. No.: 15/822,245

(22) Filed: Nov. 27, 2017

Related U.S. Application Data
(63) Continuation of application No. 15/653,767, filed on Jul. 19, 2017.

(51) Int. Cl. A61B 5/00; A61B 5/11; G06K 9/00; G06T 7/00

(52) U.S. Cl. CPC A61B 5/4561; A61B 5/1116; G06T 7/00; A61B 5/0022; A61B 5/1114; G06K 9/00369

(57) ABSTRACT

Embodiments of the invention are directed to computer-implemented methods, computer systems, and computer program products for monitoring the ergonomics of a user. A non-limiting example method includes capturing a reference image of the user using an image capture device. The method further includes determining a first height of an object of interest of the user using the reference image of the user. The method further includes capturing a subsequent image of the user using the image capture device. The method further includes determining a second height of the object of interest of the user using the subsequent image of the user. Based at least in part on determining that the second height is not within a first tolerance of the first height, causing the issuance of a warning that the user is not within an optimal distance from the image capture device.

### CROSS-REFERENCE TO RELATED APPLICATION

This application is a continuation of U.S. application Ser. No. 15/653,767, filed Jul. 19, 2017, the contents of which are hereby incorporated by reference in its entirety.

### BACKGROUND

The present invention generally relates to the field of computing. More specifically, the present invention relates to monitoring the posture of a person.

People who have to be in a single position (e.g., sitting) for long periods of time can be at risk of repetitive stress injuries and other musculoskeletal disorders that can develop over time and can lead to long-term disability. Human factors and ergonomics can be employed to help prevent such disorders by attempting to ensure that a person maintaining a single position has the proper posture that would prevent such injuries.

### SUMMARY

Embodiments of the present invention are directed to a computer-implemented method for monitoring the ergonomics of a user. A non-limiting example method includes capturing a reference image of the user using an image capture device. The method further includes determining a first height of an object of interest of the user using the reference image of the user. The method further includes capturing a subsequent image of the user using the image capture device. The method further includes determining a second height of the object of interest of the user using the subsequent image of the user. Based at least in part on determining that the second height is not within a first tolerance of the first height, causing the issuance of a warning that the user is not within an optimal distance from the image capture device.

Embodiments of the present invention are directed to a computer system for monitoring the ergonomics of a user. The computer system includes a memory and a processor system communicatively coupled to the memory. The processor system is configured to perform a method. A non-limiting example method includes capturing a reference image of the user using an image capture device. The method further includes determining a first height of an object of interest of the user using the reference image of the user. The method further includes capturing a subsequent image of the user using the image capture device. The method further includes determining a second height of the object of interest of the user using the subsequent image of the user. Based at least in part on determining that the second height is not within a first tolerance of the first height, causing the issuance of a warning that the user is not within an optimal distance from the image capture device.

Embodiments of the present invention are directed to a computer program product for monitoring the ergonomics of a user. The computer program product includes a computer-readable storage medium having program instructions embodied therewith. The program instructions are readable by a processor system to cause the processor system to perform a method. A non-limiting example method includes capturing a reference image of the user using an image capture device. The method further includes determining a first height of an object of interest of the user using the reference image of the user. The method further includes capturing a subsequent image of the user using the image capture device. The method further includes determining a second height of the object of interest of the user using the subsequent image of the user. Based at least in part on determining that the second height is not within a first tolerance of the first height, causing the issuance of a warning that the user is not within an optimal distance from the image capture device.

Additional features and advantages are realized through techniques described herein. Other embodiments and aspects are described in detail herein. For a better understanding, refer to the description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the embodiments of the invention are apparent from the following detailed description taken in conjunction with the accompanying drawings in which:

FIG. 1 depicts an overview of one example of a proper ergonomic position;

FIG. 2 depicts a flow diagram illustrating a methodology according to embodiments of the invention;

FIG. 3 depicts a computer system capable of implementing hardware components according to embodiments of the invention;

FIG. 4 depicts a diagram of a computer program product according to embodiments of the invention;

FIG. 5A depicts a diagram of a reference image of a user according to embodiments of the invention;

FIG. 5B depicts a diagram of a subsequent image of a user who is not in a proper ergonomic position according to embodiments of the invention;

FIG. 5C depicts a diagram of a subsequent image of a user who is not in a proper ergonomic position according to embodiments of the invention; and

FIG. 6 depicts a flow diagram illustrating a methodology according to embodiments of the invention; and

The diagrams depicted herein are illustrative. There can be many variations to the diagram or the operations described therein without departing from the spirit of the invention. For instance, the actions can be performed in a differing order or actions can be added, deleted or modified. In addition, the term “coupled” and variations thereof describes having a communications path between two elements and does not imply a direct connection between the elements with no intervening elements/connections between them. All of these variations are considered a part of the specification.

In the accompanying figures and following detailed description of the disclosed embodiments, the various elements illustrated in the figures are provided with two or three digit reference numbers. With minor exceptions, the leftmost digit(s) of each reference number correspond to the figure in which its element is first illustrated.

### DETAILED DESCRIPTION

Various embodiments of the present invention will now be described with reference to the related drawings. Alternate embodiments can be devised without departing from the scope of this invention. Various connections might be set forth between elements in the following description and in the drawings. These connections, unless specified otherwise, can be direct or indirect, and the present description is not intended to be limiting in this respect. Accordingly, a coupling of entities can refer to either a direct or an indirect connection.

Additionally, although a detailed description of a system is presented, configuration and implementation of the teachings recited herein are not limited to a particular type or configuration of device(s). Rather, embodiments are capable of being implemented in conjunction with any other type or configuration of devices and/or environments, now known or later developed.

Furthermore, although a detailed description of usage with specific devices is included herein, implementation of the teachings recited herein are not limited to embodiments described herein. Rather, embodiments are capable of being implemented in conjunction with any other type of electronic device, now known or later developed.

At least the features and combinations of features described in the immediately present application, including the corresponding features and combinations of features depicted in the figures amount to significantly more than implementing a method of remote monitoring of sensors. Additionally, at least the features and combinations of features described in the immediately following paragraphs, including the corresponding features and combinations of features depicted in the figures go beyond what is well understood, routine and conventional in the relevant field(s).

As stated above, computer users are particularly at risk of having bad posture. Studies have shown that almost 31 million days of work were lost in the year 2014 due to back, neck, and muscle problems. Such injuries can be mitigated to an extent with proper posture.

Ergonomics as it relates to computer users involves the proper posture of a user. This involves several factors, such as the monitor of a user being positioned at the correct height, the keyboard being positioned at a correct height, the user's head and torso being positioned correctly.

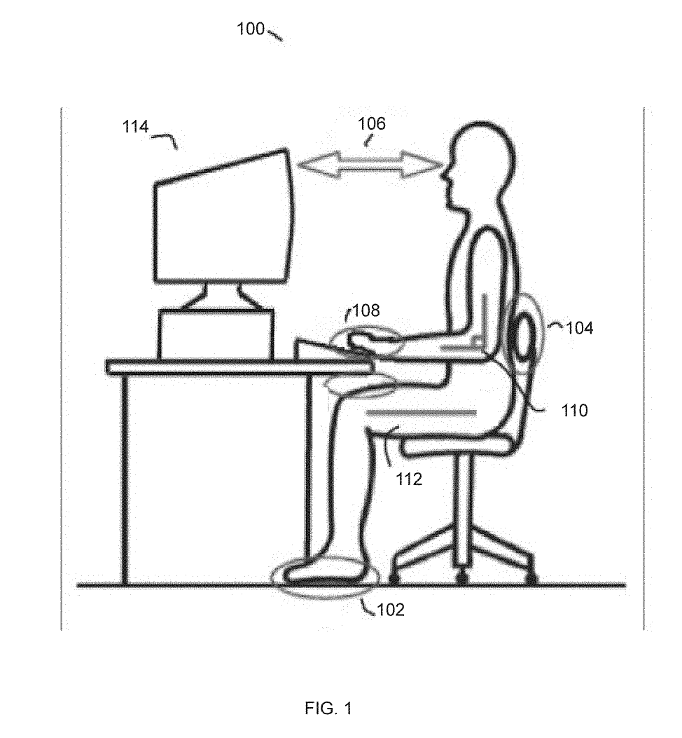

With reference to FIG. 1, an exemplary proper ergonomic position of a user is illustrated in diagram 100. Ideally, a user has several factors that determine if a user is in a proper ergonomic position. For example, the user's feet 102 should be flat on the floor. A footrest (not shown) can be provided if that is not possible. The user's chair should have back support 104 at the user's lower back. The distance between the user's eyes and the computer monitor 114 should be at an arm's length 106. The user's wrist should be level with the user's forearm (108). The bend in the user's elbow 110 should be approximately 90 degrees. The user's thighs 112 should be parallel to the ground. Other factors also can be taken into consideration.

An issue that can occur with a user's posture is that it can change during the course of a day. For example, a user may start the day with proper posture, but begin slouching later in the day, possibly causing pain in the user's lower back. Existing technologies rely on wearable sensors placed on the user's body or sensor equipped cushions. There are a variety of issues with those solutions. For example, sensors can be relatively expensive. In addition, they can be uncomfortable, resulting in a user not wanting to use them. In addition, existing sensors only account for upright posture and do not account for other body positions, such as standing or the position used by a cello player.

Embodiments of the present invention address the above-described issues by using a novel method and system to monitor the posture of a user. An image-capturing device is used to determine the posture of the user and alert the user. The images can be compared to reference issues made of the user to determine if the user has deviated from proper posture.

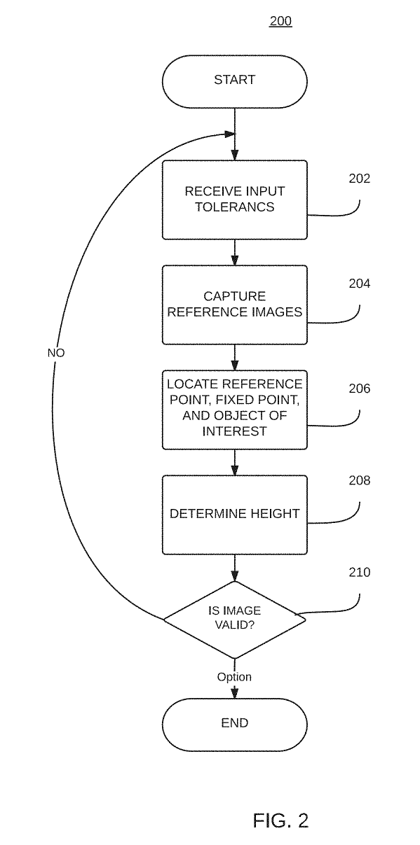

A flowchart illustrating method 200 is presented in FIG. 2. Method 200 is merely exemplary and is not limited to the embodiments presented herein. Method 200 can be employed in many different embodiments or examples not specifically depicted or described herein. In some embodiments, the procedures, processes, and/or activities of method 200 can be performed in the order presented. In other embodiments, one or more of the procedures, processes, and/or activities of method 200 can be combined or skipped. In one or more embodiments, method 200 is performed by a processor as it is executing instructions.

Input tolerances are received for both a vertical and horizontal distances (block 202). In some embodiments, these tolerances can have a default value. In some embodiments, these tolerances can be set by a user.

Reference images are captured of the user (block 204). These reference images are captured of the user at the ideal posture. In some embodiments, the user is monitored to determine when the user is in an ideal posture. Thereafter, the reference images are captured. The reference image can be captured by one of a variety of different image capturing devices. In some embodiments, a webcam can be used. In other embodiments, any type of camera or video camera can be used to capture the reference images. The image-capturing device is typically at a fixed point so that the reference image can later be compared to other images, as described in further detail below.

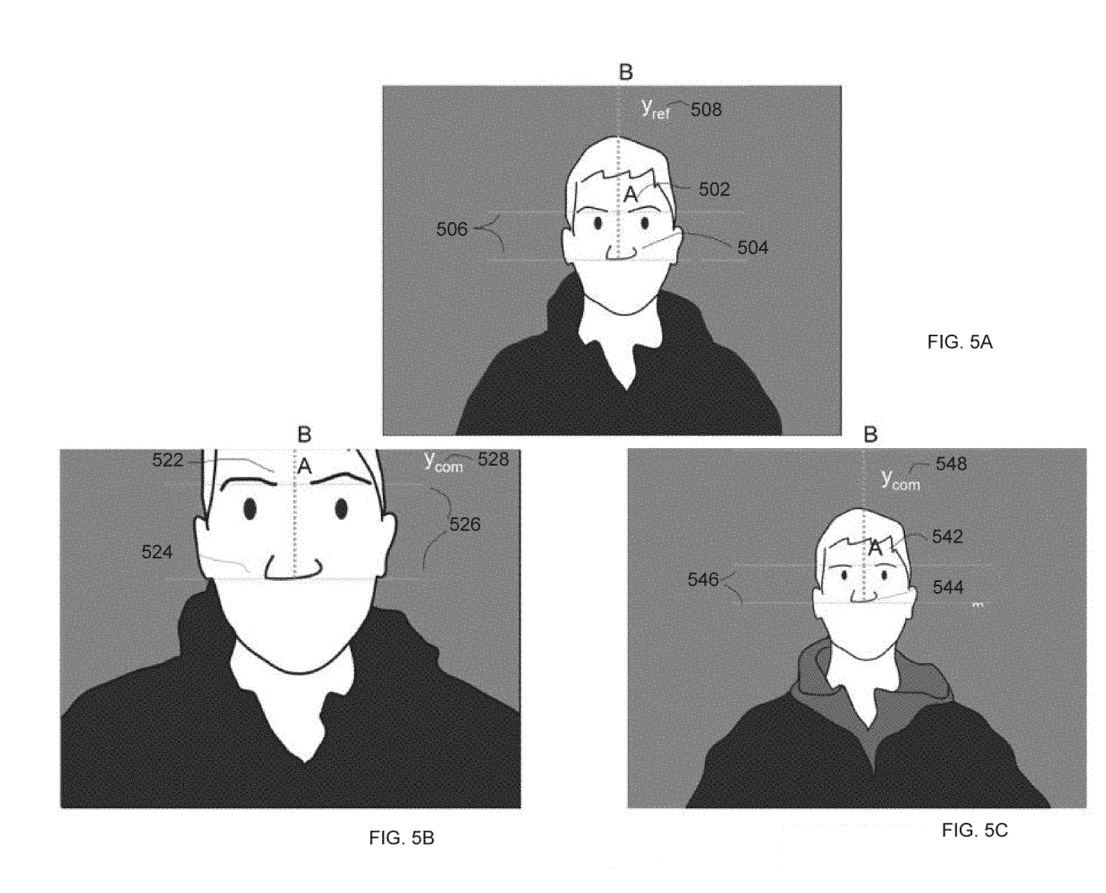

A reference point A, a fixed point B, and an object of interest O are located on the reference image (block 206). These can be seen more easily with reference to FIG. 5A through 5C, described in further detail below. Thereafter, the height (Href) of the object of interest is determined (block 208). Finding the location of reference point A, fixed point B, and object of interest O can be accomplished in one of a variety of different manners. In some embodiments, machine-learning techniques can be used to find these points on an image captured by the image capture device. In FIG. 5A, a reference image is illustrated of an exemplary user. The reference point A is defined as the eyebrows 502. The object of interest O is defined as his nose 504. The height (Href) (506) in this case is defined as the distance between the user's nose 504 and his eyebrows 502. It should be understood that any feature of the user can be used to determine the reference point and the object of interest. For some users, other features may be used instead, such as the height of the user's eyeglasses, length of the neck, length of the ear, and the like. The distance between the fixed point B and the reference point A is also determined. With reference to FIG. 5A, the fixed point is the top of the frame. Yref (508) is the distance between point A and point B. It should be understood that other fixed points can be used.

Returning to FIG. 2, it is determined if a reference image is valid (block 210). If not, the process stops (block 212) and can be re-started again later. A variety of conditions can determine if the reference image is valid. For example, the exposure of the image should be such that the features of the user's face are visible and can be measured. The image should be in focus and the features of interest in the user's face should be in frame. If the reference image is invalid, the user can be notified to make changes to the image-capturing device.

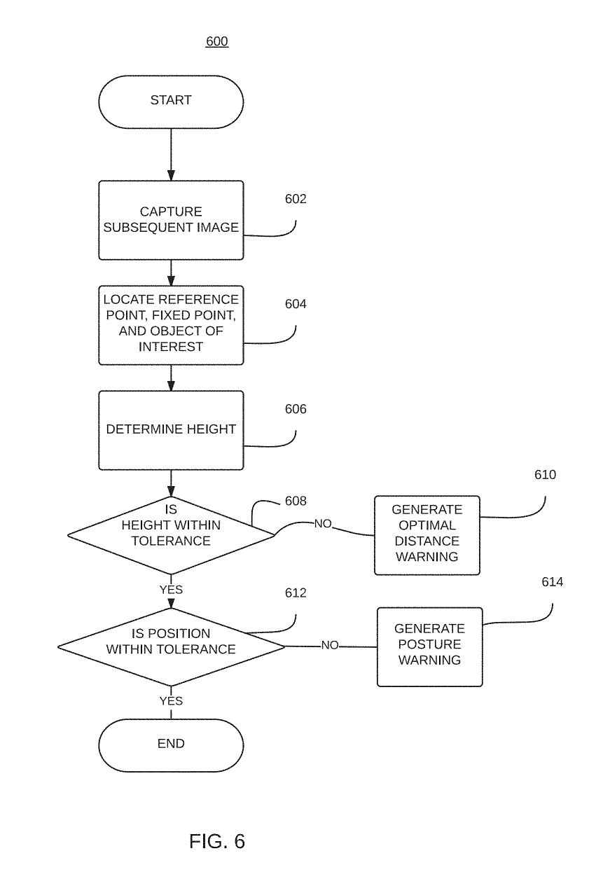

A flowchart illustrating method 600 is presented in FIG. 6. Method 600 is merely exemplary and is not limited to the embodiments presented herein. Method 600 can be employed in many different embodiments or examples not specifically depicted or described herein. In some embodiments, the procedures, processes, and/or activities of method 600 can be performed in the order presented. In other embodiments, one or more of the procedures, processes, and/or activities of method 600 can be combined or skipped. In one or more embodiments, method 600 is performed by a processor as it is executing instructions.

Method 600 depicts the operations undertaken by one or more embodiments after a reference image has been taken using a method such as method 200. A subsequent image is captured (block 602).

A reference point A, a fixed point B, and an object of interest O are located on the subsequent image (block 604). These can be seen more easily with reference to FIG. 5B through 5C, described in further detail below. Finding the location of reference point A, fixed point B, and object of interest O can be accomplished in one of a variety of different manners. In some embodiments, machine-learning techniques can be used to find these points on an image captured by the image capture device. Thereafter, the height (Hcom) of the object of interest is determined (block 606).

In FIG. 5B, a subsequent image is illustrated of an exemplary user. The reference point A is defined as the eyebrows 522. The object of interest is the user's nose 524. The height (Hcom) (526) in this case is defined as the distance between the user's nose and his eyebrows. It should be understood that any feature of the user can be used to determine the reference point and the object of interest. For some users, other features may be used instead, such as the height of the user's eyeglasses, length of the neck, length of the ear, and the like. The distance between the fixed point B and the reference point A is also determined. With reference to FIG. 5B, the fixed point is the top of the frame. Ycom (528) is the distance between point A and point B.

In FIG. 5C, a subsequent image is illustrated of an exemplary user. The reference point A is defined as the eyebrows 542. The object of interest is the user's nose 544. The height (Hcom) (546) in this case is defined as the distance between the user's nose and his eyebrows. It should be understood that any feature of the user can be used to determine the reference point and the object of interest. For some users, other features may be used instead, such as the height of the user's eyeglasses, length of the neck, length of the ear, and the like. The distance between the fixed point B and the reference point A is also determined. With reference to FIG. 5B, the fixed point is the top of the frame. Ycom (548) is the distance between point A and point B.

Referring back to FIG. 6, the height and position in the subsequent image are compared to those of the reference image (block 608). It is determined if the absolute value of the difference between Href and Hcom is less than the tolerance. If it is, then the optimal distance is present. If it is greater than the tolerance, then there is a violation of the optimal distance (block 610). In either situation, a warning or alert can be presented to the user to let them know of the problem. The warning or alert can be tactile (e.g., a signal transmitted to a watch or bracelet worn by the user), visual (e.g., a warning displayed on a computer monitor), audible (e.g., a warning tone sounded through speakers or headphones coupled to a computer), or a combination thereof. The warning or alert can be generated in any one of a variety of different manners known in the art.

The absolute value of the difference between Ycom and Yref is compared to the tolerance (612). If it is greater, then there is a posture violation (block 614). Otherwise, method 600 ends. In some embodiments, method 600 can be run in a periodic manner to ensure that the user's posture remains within ergonomic norms.

As described above, FIG. 5A illustrates a reference image. FIG. 5B illustrates a user who is too close to the imaging device. It can be seen that, in FIG. 5B, Ycom is much less than Yref and Hcom is much less than Href. FIG. 5C illustrates a user who is too far from the imaging device. It can be seen that, in FIG. 5B, Ycom is much greater than Yref while Hcom is much greater than Href. There can be other situations (not illustrated), where the height Hcom is correct. But the position, Ycom, is not within tolerance.

If the height is within tolerance, this indicates that the user is the correct distance from the imaging device, because the user's face is the same size in both the reference image and the newly taken image. The position not being within tolerance while the height is within tolerance indicates that the user's head is not in the correct position, which can be indicative of the user slouching or otherwise being in a non-optimal position.

Using one or more embodiments of the present invention in the above described manner allows a user to continuously or periodically monitor his ergonomics at a certain location. This can include a user's computer workstation, where a user may spend a large portion of his time. Alerting the user when he does not have proper ergonomics can result in the user improving his ergonomics and posture, resulting in long-term health benefits.

Embodiments can be used in other environments. One or more embodiments can be placed in moving vehicles. Similar issues to those described above with respect to a computer workstation can exist for people who sit for long periods of time in a vehicle, such as a truck driver, bus driver, airplane pilot, and the like. Any person who is required to sit for long period of time can benefit from embodiments of the invention. Musicians, for example, can utilize proper ergonomics to ensure their technique is smooth. For some musicians, a proper position might not be an upright position, due to the size or bulk of the instrument being played. Embodiments of the present invention can still monitor and alert users that their posture is not optimal even if the optimal posture is not upright.

It should be understood that that embodiments are not restricted to a seated user. Some people are required to stand for long periods of time. A periodic ergonomic check of their posture using one or more embodiments can help preserve the health and well-being of such users.

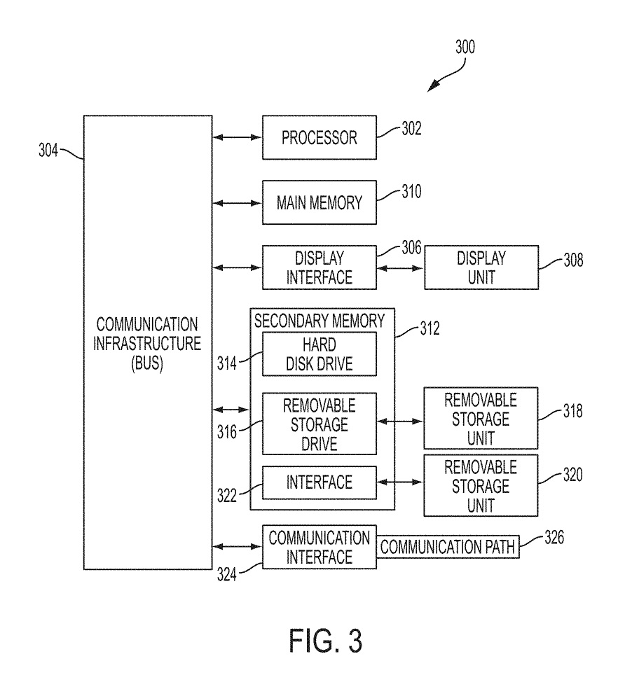

FIG. 3 depicts a high-level block diagram of a computer system 300, which can be used to perform the above-described methods in one or more embodiments. More specifically, computer system 300 can be used to implement hardware components of systems capable of performing methods described herein. Although one exemplary computer system 300 is shown, computer system 300 includes a communication path 326, which connects computer system 300 to additional systems (not depicted) and can include one or more wide area networks (WANs) and/or local area networks (LANs) such as the Internet, intranet(s), and/or wireless communication network(s). Computer system 300 and additional system are in communication via communication path 326, e.g., to communicate data between them.

Computer system 300 includes one or more processors, such as processor 302. Processor 302 is connected to a communication infrastructure 304 (e.g., a communications bus, crossover bar, or network). Computer system 300 can include a display interface 306 that forwards graphics, textual content, and other data from communication infrastructure 304 (or from a frame buffer not shown) for display on a display unit 308. Computer system 300 also includes a main memory 310, preferably random access memory (RAM), and can also include a secondary memory 312. Secondary memory 312 can include, for example, a hard disk drive 314 and/or a removable storage drive 316, representing, for example, a floppy disk drive, a magnetic tape drive, or an optical disc drive. Hard disk drive 314 can be in the form of a solid-state drive (SSD), a traditional magnetic disk drive, or a hybrid of the two. There also can be more than one hard disk drive 314 contained within secondary memory 312. Removable storage drive 316 reads from and/or writes to a removable storage unit 318 in a manner well known to those having ordinary skill in the art. Removable storage unit 318 represents, for example, a floppy disk, a compact disc, a magnetic tape, or an optical disc, etc. which is read by and written to by removable storage drive 316. As will be appreciated, removable storage unit 318 includes a computer-readable medium having stored therein computer software and/or data.

In alternative embodiments, secondary memory 312 can include other similar means for allowing computer programs or other instructions to be loaded into the computer system. Such means can include, for example, a removable storage unit 320 and an interface 322. Examples of such means can include a program package and package interface (such as that found in video game devices), a removable memory chip (such as an EPROM, secure digital card (SD card), compact flash card (CF card), universal serial bus (USB) memory, or PROM) and associated socket, and other removable storage units 320 and interfaces 322 which allow software and data to be transferred from the removable storage unit 320 to computer system 300.

Computer system 300 can also include a communications interface 324. Communications interface 324 allows software and data to be transferred between the computer system and external devices. Examples of communications interface 324 can include a modem, a network interface (such as an Ethernet card), a communications port, or a PC card slot and card, a universal serial bus port (USB), and the like. Software and data transferred via communications interface 324 are in the form of signals that can be, for example, electronic, electromagnetic, optical, or other signals capable of being received by communications interface 324. These signals are provided to communications interface 324 via communication path (i.e., channel) 326. Communication path 326 carries signals and can be implemented using wire or cable, fiber optics, a phone line, a cellular phone link, an RF link, and/or other communications channels.

In the present description, the terms “computer program medium,” “computer usable medium,” and “computer-readable medium” are used to refer to media such as main memory 310 and secondary memory 312, removable storage drive 316, and a hard disk installed in hard disk drive 314. Computer programs (also called computer control logic) are stored in main memory 310 and/or secondary memory 312. Computer programs also can be received via communications interface 324. Such computer programs, when run, enable the computer system to perform the features discussed herein. In particular, the computer programs, when run, enable processor 302 to perform the features of the computer system. Accordingly, such computer programs represent controllers of the computer system. Thus it can be seen from the forgoing detailed description that one or more embodiments provide technical benefits and advantages.

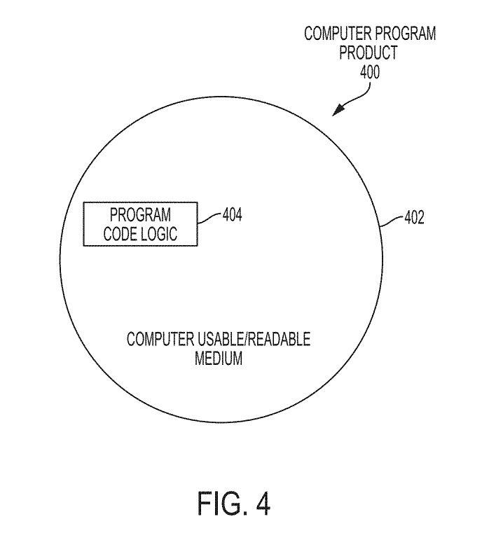

Referring now to FIG. 4 a computer program product 400 in accordance with an embodiment that includes a computer-readable storage medium 402 and program instructions 404 is generally shown.

Embodiments can be a system, a method, and/or a computer program product. The computer program product can include a computer-readable storage medium (or media) having computer-readable program instructions thereon for causing a processor to carry out aspects of embodiments of the present invention.

The computer-readable storage medium can be a tangible device that can retain and store instructions for use by an instruction execution device. The computer-readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network and/or a wireless network. The network can include copper transmission cables, optical transmission fibers, wireless transmission, routers, firewalls, switches, gateway computers, and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions for carrying out embodiments can include assembler instructions, instruction-set-architecture (ISA) instructions, machine instructions, machine dependent instructions, microcode, firmware instructions, state-setting data, or either source code or object code written in any combination of one or more programming languages, including an object-oriented programming language such as Smalltalk, C++ or the like, and conventional procedural programming languages, such as the “C” programming language or similar programming languages. The computer-readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer-readable program instructions by utilizing state information of the computer-readable program instructions to personalize the electronic circuitry, in order to perform embodiments of the present invention.

Aspects of various embodiments are described herein with reference to flowchart illustrations and/or block diagrams of methods, apparatus (systems), and computer program products according to various embodiments. It will be understood that each block of the flowchart illustrations and/or block diagrams, and combinations of blocks in the flowchart illustrations and/or block diagrams, can be implemented by computer-readable program instructions.

These computer-readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions can also be stored in a computer-readable storage medium that can direct a computer, a programmable data processing apparatus, and/or other devices to function in a particular manner, such that the computer-readable storage medium having instructions stored therein includes an article of manufacture including instructions which implement aspects of the function/act specified in the flowchart and/or block diagram block or blocks.

The computer-readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams can represent a module, segment, or portion of instructions, which includes one or more executable instructions for implementing the specified logical function(s). In some alternative implementations, the functions noted in the block can occur out of the order noted in the figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

The corresponding structures, materials, acts, and equivalents of all means or step plus function elements in the claims below are intended to include any structure, material, or act for performing the function in combination with other claimed elements as specifically claimed. The descriptions presented herein are for purposes of illustration and description, but is not intended to be exhaustive or limited. Many modifications and variations will be apparent to those of ordinary skill in the art without departing from the scope and spirit of embodiments of the invention. The embodiment was chosen and described in order to best explain the principles of operation and the practical application, and to enable others of ordinary skill in the art to understand embodiments of the present invention for various embodiments with various modifications as are suited to the particular use contemplated.

What is claimed is:

1. A computer-implemented method for monitoring the ergonomics of a user via a system comprising an image capture device, a memory, and a processor communicatively coupled to the memory and to the image capture device, the method comprising:
capturing, by the image capture device, a reference image of the user;
executing, by the processor, a machine learning technique on the reference image to identify, within a frame of the reference image, an object of interest located on the user and a reference point located on the user;
determining, by the processor, a first height of the object of interest of the user using the reference image of the user, wherein the first height comprises a distance between a position of the object of interest with respective to the frame of reference image and a position of the reference point with respective to the frame of reference image;
capturing, by the image capture device, a subsequent image of the user;
executing, by the processor, a machine learning technique on the subsequent image to identify, within a frame of the subsequent image, the object of interest located on the user and the reference point located on the user;
determining, by the processor, a second height of the object of interest of the user using the subsequent image of the user, wherein the second height comprises a distance between a position of the object of interest with respective to the frame of the subsequent image and a position of the reference point with respect to the frame of the subsequent image; and
based at least in part on determining that the second height is not within a first tolerance of the first height, causing the issuance of a warning that the user is not within an optimal distance from the image capture device.

2. The computer-implemented method of claim 1 further comprising:
determining, by the processor, a first position of the reference point in the reference image of the user;
determining, by the processor, a second position of the reference point in the subsequent image of the user; and
upon determination that the second position is not within a second tolerance of the first position, causing the issuance of a warning that the user does not have an optimum posture.

3. The computer-implemented method of claim 2, wherein:
determining the first position comprises determining a distance between the reference point in the reference image and a fixed point in the reference image; and
determining the second position comprises determining a distance between the reference point in the subsequent image and a fixed point in the subsequent image.

4. The computer-implemented method of claim 3, wherein:
the fixed point in the reference image is a point on the frame of the reference image; and
the fixed point in the subsequent image is a point on the frame of the subsequent image.

5. The computer-implemented method of claim 1, wherein:
determining the first height comprises determining a distance between the reference point in the reference image and an object of interest in the reference image; and
determining the second position comprises determining a distance between the reference point in the subsequent image and an object of interest in the subsequent image.

6. The computer-implemented method of claim 1, further comprising:
receiving, by the processor, an input of the first tolerance.

7. The computer-implemented method of claim 1, further comprising:
upon a determination, by the processor, that the reference image is not a valid image, performing another capture of the reference image.
